(19) United States
(12) Patent Application Publication
(10) Pub. No.: US 2018/0280671 A1
(43) Pub. Date: Oct. 4, 2018

(54) DEVICE FOR TREATING VENOUS INCOMPETENCE, AND RELATED METHODS

(71) Applicant: Christopher Parr, Farmington, UT (US)

(72) Inventor: Christopher Parr, Farmington, UT (US)

(21) Appl. No.: 15/941,801

(22) Filed: Mar. 30, 2018

Related U.S. Application Data
(60) Provisional application No. 62/480,528, filed on Apr. 2, 2017.

(51) Int. Cl. A61M 29/02; A61B 17/12

(52) U.S. Cl. CPC A61M 29/02; A61B 17/1204; A61B 2017/00893; A61B 17/12131; A61B 2017/1205; A61B 17/12109

(57) ABSTRACT

The present disclosure relates to the treatment of venous incompetence. More specifically, the present disclosure reduces the volume of chemical needed to treat incompetent veins by distorting the luminal vessel. A device for treating incompetent veins comprises a catheter disposed within a sheath; and an expansion element disposed within the sheath in a compressed state and attached to the catheter, the expansion element configured such that when the sheath is retracted within a luminal vessel the expansion element expands to contact a wall of the luminal vessel, wherein expansion of the expansion element is configured to reduce a cross-sectional area of the luminal vessel. Related methods of venous incompetence are also disclosed.

### CROSS-REFERENCE TO RELATED APPLICATION

This application claims the benefit of U.S. Provisional Patent Application Ser. No. 62/480,528, filed Apr. 2, 2017, and titled “ENDOLUMINAL CATHETER,” the disclosure of which application is hereby incorporated herein in its entirety by this reference.

### TECHNICAL FIELD

Embodiments of the disclosure relate generally to devices for treating lower extremity venous incompetence and associated varicose veins and to related methods. More particularly, embodiments of the disclosure relate to devices for delivering a therapeutic agent to a patient, the device including a catheter having expandable members coupled thereto, the expandable members and configured to expand in the vascular system of the patient, the catheter including perforations for delivering the therapeutic agent to the patient, and to related methods of treating a patient.

### BACKGROUND

Many people in the United States suffer from great saphenous vein (GSV) incompetence, which may give rise to lower limb swelling, pain, ulceration, and/or symptomatic “varicose veins.” In addition to the great saphenous vein, individuals may suffer from incompetent veins and/or varicose veins at other locations of their body. Varicose veins typically occur in the legs where the valves that assist in pumping blood against gravity back to the heart become incompetent, causing reflux of in the venous vessels of the lower extremity. In the past, treatment of venous incompetence and/or varicose veins includes surgical ligation or stripping. More modern, less invasive methods include laser or radiofrequency (RF) catheter-based ablation or the truncal vessels and/or associated varicosities. Sclerosant therapy and glue-based treatments are also used to treat venous incompetence and/or varicose veins.

Although treatments for varicose veins are plentiful and effective, the thermal ablation techniques (also referred to as thermoablative techniques) often require the use of local tumescent anesthesia. The use of tumescent anesthesia increases procedure duration increases patient discomfort during the treatment procedure. One possible alternative to using the tumescent anesthetic that is required when using the thermal ablation techniques is sclerotherapy. One problem with chemical sclerosants used for schlerotherapy is that the sclerosants are dispersed into the liquid blood environment to effectuate treatment. However, it is difficult to control the dispersed sclerosant in the blood and the sclerosants can be unpredictable in their delivery. Complications associated with misdelivery of sclerosing agents include negative side effects, such as ulcers or blood clots, or even potential toxicity when the sclerosing agents are used in large volumes.

In addition, current techniques for treating varicose veins often depend on causing vein spasming and/or external compression to minimize the lumen (vessel) size in the area of treatment. Such treatment methods may allow for areas of endothelium to persist in untreated undulations of the endothelia surface that are formed during the spasming of the vein, or in areas of the vein that may not receive an adequate amount of sclerosant or other chemicals (e.g., cyanoacrylate glue) during treatment. The untreated endothelium cells may serve as a nidus (i.e., an origin) to promote re-endothelialization or re-canalization of the treated site.

Most treatment methods include introducing a catheter into the vein where treatment is to be performed. The catheter may include a laser fiber or a radiofrequency unit to thermally ablate the vein wall. Other catheters dispense glue or mechanically abrade the endothelium to cause spasming and to potentiate endothelia damage with sclerosant agents. Catheters may also include expandable balloons to isolate the segment of vein to be treated with sclerosant.

### SUMMARY

This summary of the present disclosure is not intended to describe each illustrated embodiment or every possible implementation. The present disclosure relates to a non-thermal and non-tumescent method for treating incompetent veins and/or varicose veins and a treatment device that is used with the method. The volume of sclerosant used to treat the incompetent veins may be reduced by distorting the shape of the lumen of the luminal vessel. In addition, distorting the lumen reduces the flow of blood through the treatment location of the vessel and minimizes (e.g., reduces) dilution of the sclerosant caused by inflowing blood and increases the effectiveness of the sclerosant. The devices include at least one expansion element to distort the vessel and the lumen thereof after the device has been inserted into the luminal vessel at the treatment location. The at least one expansion element may be configured to expand and distort the cross-sectional area of the luminal vessel. In some embodiments, the luminal vessel is distorted to have a substantially flattened (e.g., rectangular) or an elongated elliptical cross-sectional shape. The at least one expansion element may comprise a material such that a cross-sectional area of the luminal vessel is reduced and a perimeter of the luminal vessel proximate to the at least one expansion element is increased (i.e., a surface area of the luminal vessel is increased while the cross-sectional area proximate the at least one expansion element is reduced).

Additional features and advantages of embodiments of the disclosure will be set forth in the description which follows, and in part will be obvious from the description, or may be learned by the practice of such embodiments. The features and advantages of the disclosure may be realized and obtained by means of the instruments and combinations particularly pointed out in the appended claims. These and other features will become more fully apparent from the following description and appended claims, or may be learned by the practice of embodiments of the disclosure as set forth hereinafter.

Embodiments disclosed herein include devices and methods for treating venous incompetence and associated varicose veins. For example, in accordance with one embodiment, a device for treating venous incompetence and associated varicose veins comprises a catheter disposed within a sheath, and an expansion element disposed within the sheath in a compressed state and attached to the catheter, the expansion element configured such that when the sheath is retracted within a luminal vessel the expansion element expands to contact and laterally deform a wall of the luminal vessel to reduce a cross-sectional area of the luminal vessel.

In additional embodiments, a device for treating venous incompetence comprises a catheter having perforations in a wall thereof, the perforations located at a distal end of the catheter, and an expansion element comprising a first expansion element and a second expansion element attached to the catheter and housed within the sheath in an unexpanded configuration, the first expansion element and the second expansion element configured to expand to contact a wall of a luminal vessel in which the device is placed, wherein contact of the wall of the luminal vessel by the first expansion element and the second expansion element reduces a cross-sectional area of the luminal vessel.

In further embodiments, a method of treating venous incompetence comprises inserting a device into a luminal vessel, the device comprising a catheter disposed within a sheath, and an expansion element disposed within the sheath in a compressed state. The method further comprises placing the catheter at a treatment location, retracting the sheath to release the expansion element and contact an interior of the luminal vessel to distort the luminal vessel and reduce a cross-sectional area thereof, and passing a chemical through perforations in the catheter to treat the luminal vessel.

In yet further embodiments, a method of treating venous incompetence comprises inserting a catheter into a luminal vessel to a treatment location, distorting the luminal vessel to reduce a cross-sectional area thereof at the treatment location, and introducing a chemical through the catheter to the reduced cross-sectional area of the luminal vessel.

### BRIEF DESCRIPTION OF THE DRAWINGS

FIG. 1A is a simplified perspective view of a treatment device, in accordance with embodiments of the disclosure;

FIG. 1B is a simplified perspective view of the treatment device of FIG. 1A in a partially retracted configuration, in accordance with embodiments of the disclosure;

FIG. 1C is a simplified cross-sectional view of the treatment device of FIG. 1A in a retracted configuration;

FIG. 1D is a simplified cross-sectional view of a luminal vessel in which the device FIG. 1A is inserted and in an expanded configuration;

FIG. 2A is a simplified perspective view of a device, in accordance with other embodiments of the disclosure;

FIG. 2B is a simplified cross-sectional view of the device of FIG. 2A;

FIG. 2C is a simplified cross-sectional view of the device of FIG. 2A;

FIG. 2D is a simplified cross-sectional view of an adapter, in accordance with embodiments of the disclosure; and

FIG. 3A through FIG. 3C are a respective perspective view, plan view, and cross-sectional view of a device for treating varicose veins, in accordance with embodiments of the disclosure;

FIG. 4 is a simplified flow diagram illustrating a method of treating a patient with a device, in accordance with embodiments of the disclosure.

Although embodiments of the disclosure are amenable to various modifications and alternative forms, specifics thereof have been shown by way of example in the drawings and will be described in detail such that the disclosure will be thorough and complete, and will fully convey the scope of the disclosure to those skilled in the art. However, it should be understood, that the intention is not to limit the disclosure to particular embodiments described. On the contrary, the intention is to cover all modifications, equivalents, and alternatives falling within the spirit and scope of the disclosure as defined by the appended claims and legal equivalents thereof.

### DETAILED DESCRIPTION

Illustrations presented herein are not meant to be actual views of any particular material, component, or system, but are merely idealized representations that are employed to describe embodiments of the disclosure.

The following description provides specific details, such as material types, material thicknesses, and processing techniques in order to provide a thorough description of embodiments described herein. However, a person of ordinary skill in the art will understand that the embodiments disclosed herein may be practiced without employing these specific details. Indeed, the embodiments may be practiced in conjunction with conventional fabrication techniques employed in the industry. In addition, the description provided herein does not form a complete description a device for treating venous incompetence and/or varicose veins or a complete description of a method of treating venous incompetence and/or varicose veins with the device. Only those acts and structures necessary to understand the embodiments described herein are described in detail below. Additional acts to form a complete device for treating venous incompetence and/or varicose veins or a method of treating venous incompetence and/or varicose veins may be performed by conventional techniques.

As used herein, the term “venous incompetence” means and includes a condition where internal walls of veins (e.g., leg veins) are deteriorated and small valves thereof are defective. Incompetent veins may include defective valves, causing the blood to flow backwards, such as toward the foot where the incompetent vein is located in the leg. The terms “incompetent vein(s)” and “venous incompetence” may be used interchangeably herein. Varicose veins may be associated with venous incompetence.

As used herein, the term “luminal vessel” means and includes a vein, an artery, or any vessel through which blood may flow in a patient. A catheter that may be inserted into a luminal vessel may comprise, for example, an arterial catheter, a venous catheter, or both.

As used herein, “distortion” of a vessel means and includes changing a cross-sectional shape of the vessel. In some instances, distortion of a vessel results in a different cross-sectional area of the vessel and stretching of walls of the luminal vessel to decrease the cross-sectional area thereof.

As used herein, the term “perforations” means and includes cavities, apertures, openings, permeable portions, through which one material may flow from one side of a structure to another side of the structure. For example, a catheter including perforations may include apertures therein, wherein fluid may pass from a location internal to the catheter to a location external to the catheter. In addition, a catheter including perforations may include a permeable material through which fluid may pass from a location internal to the catheter to a location external to the catheter.

Embodiments of the disclosure relate to vein ablation. Among the known prior art there are many treatment methods for varicose veins. Currently, the most popular treatment methods include the use of a heat source, such as a laser or a radiofrequency laser, to ablate the luminal vessel. However, due to the discomfort of ablation, tumescent local anesthesia (“TLA”) is required during such treatments. Some of the problems with this method is that the use of TLA increases the procedure duration and discomfort of the patient. In addition, the procedure requires dedicated proprietary and somewhat expensive equipment to perform.

New procedures are moving away from the use the TLA to reduce the cost, duration, and discomfort of the procedure. Another method includes injecting a chemical, for example, sclerosant, at the treatment location using a catheter. This method may be less effective than ablation. In addition, the presence of the chemical may cause the luminal vessel wall to spasm causing pockets to form. These pockets may prevent the chemical from treating the walls of the vessels and may reduce the effectiveness of the treatment. In addition, the pockets may result in untreated areas of endothelium, which may persist in the pockets formed by the spasming of the vessel or in areas of the vessel that do not receive an adequate amount of the treatment fluid. Yet another method includes use of a device that, after placement thereof, expands and rotates to mechanically damage the walls of the vessel and injects a fluid causing the luminal vessel to retract. But, this method does not reduce the volume of chemical required to perform the procedure.

According to embodiments disclosed herein, a less invasive alternative treatment using chemicals, such as scleroscant, is described. The methods described herein may reduce the volume required to treat varicose veins (e.g., to close the great saphenous vein and other truncal veins) compared to conventional treatment methods. In addition, the methods and apparatuses described herein may reduce or even prevent spasming of the incompetent veins and varicose veins during treatment, which may reduce an amount of untreated areas, such as pockets including endothelium that are formed during spasming caused by conventional methods of treating venous incompetence. In some embodiments, a device for treating venous incompetence includes a catheter disposed in a sheath. The sheath may be configured to be removed after placement of the catheter in a patient. The catheter may include one or more perforations therein configured to receive a treatment fluid and deliver the treatment fluid to a luminal vessel. The device may further include at least one expansion element disposed between an outer diameter of the catheter and an inner diameter of the sheath. When the sheath is moved relative to the catheter, such as along a longitudinal axis of the catheter, the at least one expansion element may be configured to expand. Expansion of the at least one expansion element may distort the vein in which the catheter is disposed to exhibit a relatively smaller cross-sectional area than prior to expansion of the expansion element. Accordingly, the expansion element may be configured to expand in a luminal vessel of a patient and distort a cross-sectional shape of the luminal vessel. In some embodiments, the cross-sectional shape of the luminal vessel may be rectangular or elliptical responsive to contact with the expansion element. In addition to reducing the cross-sectional area of the luminal vessel, expansion of the expansion element may stretch portions of the luminal vessel wall, which may smooth the surfaces thereof, eliminating wrinkles or pockets where endothelial cells would otherwise be protected from a treatment fluid.

In use and operation, the device may be inserted into the luminal vessel of a patient. After insertion to a desired location, the expansion element may be expanded to change the cross-sectional shape of the luminal vessel. The expansion element may be configured to reduce a cross-sectional area of the luminal vessel, decreasing a volume of the luminal vessel along a length of the expansion element in the luminal vessel. The reduced cross-sectional area and the reduced volume of the luminal vessel may reduce an amount of the treatment fluid to be administered to the luminal vessel. At the same time, the expansion element may be configured to stretch and smooth at least a portion of walls (e.g., substantially all of the walls) of the luminal vessel, which may expose substantially all of the endothelium of the luminal vessel and increase an effectiveness of treatment fluids to the vessel walls. The device includes perforations in walls of a catheter, at the distal end of the catheter, or both, for delivering a treatment fluid to the luminal vessel while the expansion element is in an expanded state. After delivery of the treatment fluid, the expansion element may be retracted and the device may be removed from the patient or moved to another location to be treated.

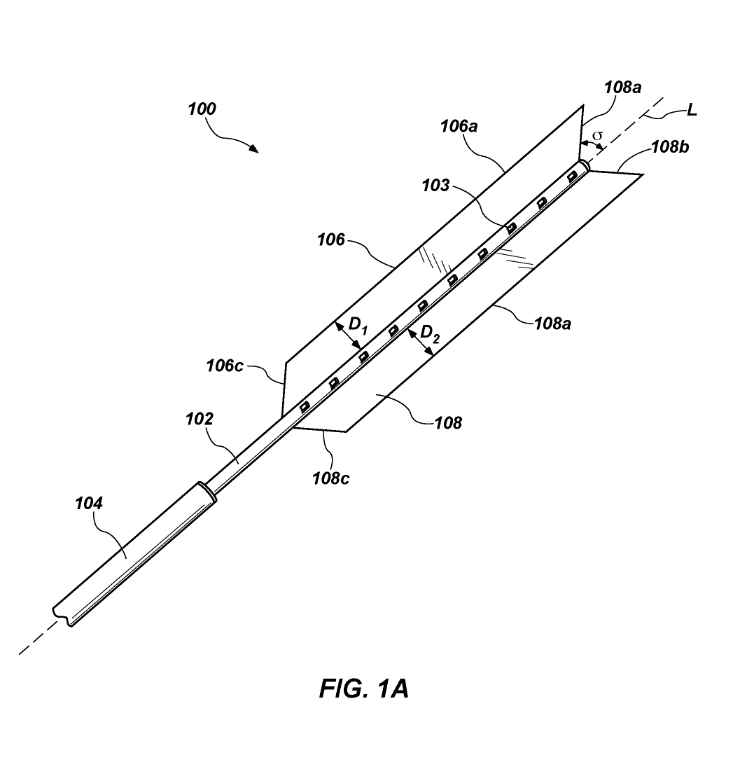

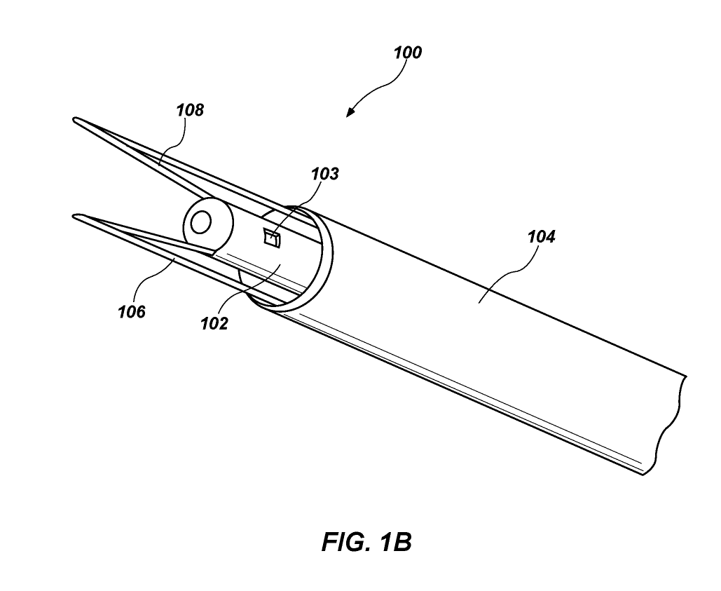

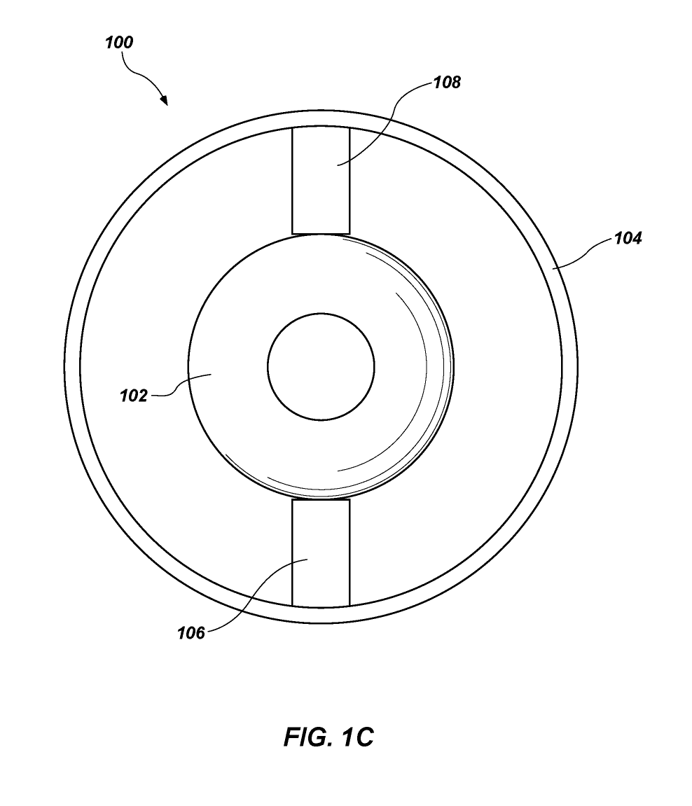

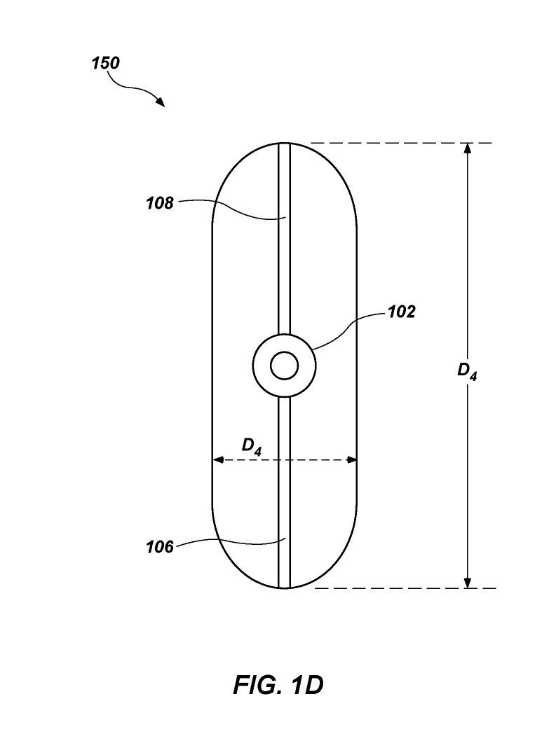

FIG. 1A is a simplified perspective view of a device 100, in accordance with embodiments of the disclosure. The device 100 includes a catheter 102 which may comprise an elongated instrument, the catheter 102 including at least one perforation 103, a sheath 104 overlying at least a portion of the catheter 102, a first expansion element 106 attached to the catheter 102, and a second expansion element 108 attached to the catheter 102.

The catheter 102 may be inserted into a patient and used to convey a fluid, such as a treatment fluid (e.g., a sclerosing agent). In some embodiments, the treatment fluid may be delivered through the at least one perforation 103, which may be formed in the wall of the catheter 102 at the distal end thereof. As used herein, the term “distal” end means and includes a location close to a patient. On the other hand, as used herein, the term “proximate” end means and includes a location close to a user, such as a medical technician. Accordingly, the distal end of the catheter 102 may be the portion of the catheter that may be inserted into the patient.

The catheter 102 may include the at least one perforation 103. In some embodiments, the catheter 102 includes a plurality of perforations, such as at least two perforations, at least 5 perforations, at least 10 perforations, at least 20 perforations, at least about 50 perforations. As will be described herein, the perforations 103 may be located at substantially a same longitudinal location of the catheter as the first expansion element 106 and the second expansion element 108. In some embodiments, the catheter 102 may include an opening at a distal end thereof, the opening configured to deliver the treatment fluid. Accordingly, the catheter 102 may include one or more perforations 103 on sidewalls thereof, an opening at the distal end thereof, or both.

In some embodiments, the catheter 102 may comprise a material that is permeable to the treatment fluid. In some such embodiments, the perforations 103 may not comprise openings in sides of the catheter 102, but may comprise a portion of the permeable material of the catheter 102. In some embodiments, the catheter 102 may comprise a permeable material and the perforations 103 may comprise the openings, as illustrated in FIG. 1A and FIG. 1B.

The catheter 102 may comprise a biocompatible polymer or an equivalent material. By way of nonlimiting example, the catheter 102 may comprise silicone rubber, nylon, polyurethane, polyethylene terephthalate (PET), latex, thermoplastic elastomers, another material, or combinations thereof.

The catheter 102 may be housed within (i.e., disposed in) the sheath 104. In other words, the sheath 104 may be configured to house the catheter 102. The sheath 104 may comprise a biocompatible polymer or an equivalent material. By way of nonlimiting example, the sheath may comprise silicone rubber, nylon, polyurethane, polyethylene terephthalate (PET), latex, thermoplastic elastomers, another material, or combinations thereof. In some embodiments, the sheath 104 comprises the same material as the catheter 102. The sheath 104 may be of a similar shape as the catheter 102 but may be larger so that it may move freely along the catheter 102.

Also, housed within (i.e., disposed in) the sheath 104 may be the first expansion element 106 and the second expansion element 108. FIG. 1B is a perspective view of the device 100 wherein the first expansion element 106 and the second expansion element 108 are illustrated as partially disposed within the sheath 104 in a compressed state and configured to expand. In FIG. 1B, the sheath 104 is illustrated as partially retracted from over the catheter 102. FIG. 1C is a simplified cross-sectional view of the device 100 when the sheath 104 is disposed over the first expansion element 106 and the second expansion element 106. With reference to FIG. 1A through FIG. 1C, the first expansion element 106 and the second expansion element 108 may be configured to expand responsive to removal of the sheath 104 from over surfaces of the first expansion element 106 and the second expansion element 108.

The first expansion element 106 and the second expansion element 108 may be made of wires comprising stainless steel, nitinol, elgiloy, a shape memory material (such as a shape memory polymer or biodegradable material (e.g., poly(alpha-hydroxy acid) such as poly-L-lactide (PLLA); poly-D-lactide (PDLA), polyglycolide (PGA), polydioxanone, polycaprolactone, polygluconate, polylactic acid-polyethylene oxide copolymers, modified cellulose, collagen, poly(hydroxybutyrate), polyanhydride, polyphosphoester, poly(amino-acids), or related copolymers)), polymers suitable for use in medical devices (e.g., nylon, polyethylene, polypropylene, etc.), another material, or combinations thereof. The material of the first expansion element 106 and the second expansion element 108 may exhibit inherent elastic properties sufficient to distort the luminal vessel responsive to expansion of the first expansion element 106 and the second expansion element 108.

The first expansion element 106 and the second expansion element 108 may extend along a longitudinal axis L of the catheter 102. With reference to FIG. 1A through FIG. 1C, in some embodiments, the first expansion element 106 may be attached to the catheter 102 and configured to expand at a location about 180° from a location from which the second expansion element 108 is configured to expand. For example, referring to FIG. 1C, when viewed in cross-section, the first expansion element 106 may be attached to the catheter 102 at a circumferential location that is about 180° from a circumferential location of a location that the second expansion element 108 is attached to the catheter 102. Accordingly, in use and operation, the first expansion element 106 may be configured to expand and contact the luminal wall at a location that is about 180° from a location of the luminal wall contacted by the second expansion element 108.

In some embodiments, the first expansion element 106 and the second expansion element 108 may exhibit the same size and shape. In some such embodiments, a surface 106a (FIG. 1A) of the first expansion element 106 may be configured to expand a distance D₁ from the catheter 102, which may be equal to about a same distance D₂ from a surface 108a (FIG. 1A) of the second expansion element 108 from the catheter 102. Stated another way, D₁ may be equal to about D₂. In other embodiments, D₁ may be different than D₂. In some embodiments, the surface 106a may be substantially parallel with the surface 108a.

The first expansion element 106 may include a leading end (i.e., a distal end) 106b (FIG. 1A) located at the distal end of the catheter 102. The second expansion element 108 may include a leading end 108b (FIG. 1A) located at the distal end of the catheter 102. In some embodiments, the leading end 106b and the leading end 108b may extend beyond a distal end of the catheter 102. In some embodiments, the leading ends 106b, 108b may extend at an angle θ relative to the longitudinal axis L of the catheter 102. In some embodiments, the angle θ at which the leading end 106b extends relative to the longitudinal axis L is about the same as the angle θ at which the leading end 108b extends relative to the longitudinal axis L. In some embodiments, forming the first expansion element 106 and the second expansion element 108 to include respective leading ends 106b, 108b may increase an effectiveness of delivery of a treatment fluid to the luminal vessels in use and operation. For example, the luminal wall may be expanded with the first expansion element 106 and the second expansion element 108 at a location extending beyond the distal end of the catheter 102 at least by the distance by which the leading ends 106b, 108b protrude beyond the distal end of the catheter 102.

In some embodiments, the perforations 103 may be circumferentially offset from a position of the first expansion element 106 and the second expansion element 108. Stated another way, the perforations 103 may be located at positions that are circumferentially offset from a location of the first expansion element 106 and the second expansion element 108. In some embodiments, the perforations 103 are located on the catheter 102 and may be circumferentially offset from the first expansion element 106 and the second expansion element by about 90°.

FIG. 1D is a simplified cross-sectional view of a luminal vessel 150 in which the device 100 has been deployed and the first expansion element 106 and the second expansion element 108 have been expanded to contact inner walls of the luminal vessel. In some embodiments, responsive to contact with the first expansion element 106 and the second expansion element 108 with the inner wall of the luminal vessel 150, the luminal vessel 150 may be distorted. The first expansion element 106 and the second expansion element 108 may be configured to reduce a cross-sectional area of the luminal vessel 150, such as by increasing a dimension of the luminal vessel 150 in a first direction and reducing the dimension of the luminal vessel 150 in a second, substantially perpendicular direction. With continued reference to FIG. 1D, a distance D₃ between first opposing walls of the luminal vessel 150 may be greater than a distance D₄ (also referred to as the minor axis dimension) between second opposing walls, located about 90° from the first opposing walls. In some embodiments, D₃ may be referred to herein as a major axis of the cross-sectional shape of the luminal vessel 105 and D₄ may be referred to herein as a minor axis of the cross-sectional shape of the luminal vessel 150. In some embodiments, a ratio of D₃:D₄ may be between about 2:1 and about 5:1, such as between about 2:1 and about 3:1, between about 3:1 and about 4:1, or between about 4:1 and about 5:1.

In some embodiments, the first expansion element 106 and the second expansion element 108 may be configured to increase the major axis of the luminal vessel 150 by a factor of at least about 2, at least about 3, at least about 4, or even at least about 5. In other words, the major axis D₃ of the luminal vessel may be increased by at least about 100%, at least about 200%, at least about 300%, or even at least about 400%. In some embodiments, the minor axis dimension D₄ may be reduced as the luminal walls extending along the major axis are stretched. In some embodiments, the first expansion element 106 and the second expansion element 108 may be configured to increase the major axis of the luminal vessel 150 until the minor axis has about a same dimension as the catheter 102. In other words, the first expansion element 106 and the second expansion element 108 may stretch the luminal walls such that the minor axis dimension D₄ is reduced to about a diameter of the catheter 102.

With continued reference to FIG. 1D, when viewed in cross-section, the first expansion element 106 and the second expansion element 108 may be configured such that the luminal vessel 150 is only contacted at two locations when expanded by the first expansion element 106 and the second expansion element 108.

Since the perforations 103 may be located on the catheter 102 at a different circumferential location than the first expansion element 106 and the second expansion element 108, the device 100 may be configured to deliver fluid directly to a stretched wall of the luminal vessel (i.e., the walls having a length of D₃). In some such embodiments, the perforations 103 may be located closer to the luminal wall than if the perforations 103 are located at substantially the same circumferential location as the first expansion element 106 and the second expansion element 108. In addition, because the first expansion element 106 and the second expansion element 108 stretch the luminal wall, the luminal wall may not include pockets that form during spasming of the luminal vessel that typically occurs during treatment with conventional devices. Accordingly, the luminal vessel may include a reduced amount of (e.g., may be substantially free of) unexposed endothelium that is not treated.

With reference to FIG. 1A, the first expansion element 106 and the second expansion element 108 may include a proximal surface 106c, 108c comprising a tapered (angled) surface. In some embodiments, the taper of the proximal surface 106c, 108c may facilitate insertion of the first expansion element 106 and the second expansion element 108 into the sheath 104 after use of the catheter 102 is completed and it is desired to retract the device 100 from the luminal vessel.

Although FIG. 1A through FIG. 1D illustrate that the device 100 includes two expansion elements 106, 108, the disclosure is not so limited. In other embodiments, the device 100 may include, for example, more expansion elements, such as three expansion elements, four expansion elements, five expansion elements, six expansion elements, etc. As one example, the device may include three expansion elements equally spaced along a circumference of the catheter 102. In some such embodiments, the expansion elements may be configured to form a substantially triangular cross-sectional shape when in an expanded configuration. As another example, the device 100 may include four expansion elements. In some such embodiments, the expansion elements located about 180° from each other may have substantially the same dimensions while expansion elements located about 90° from each other may have different dimensions.

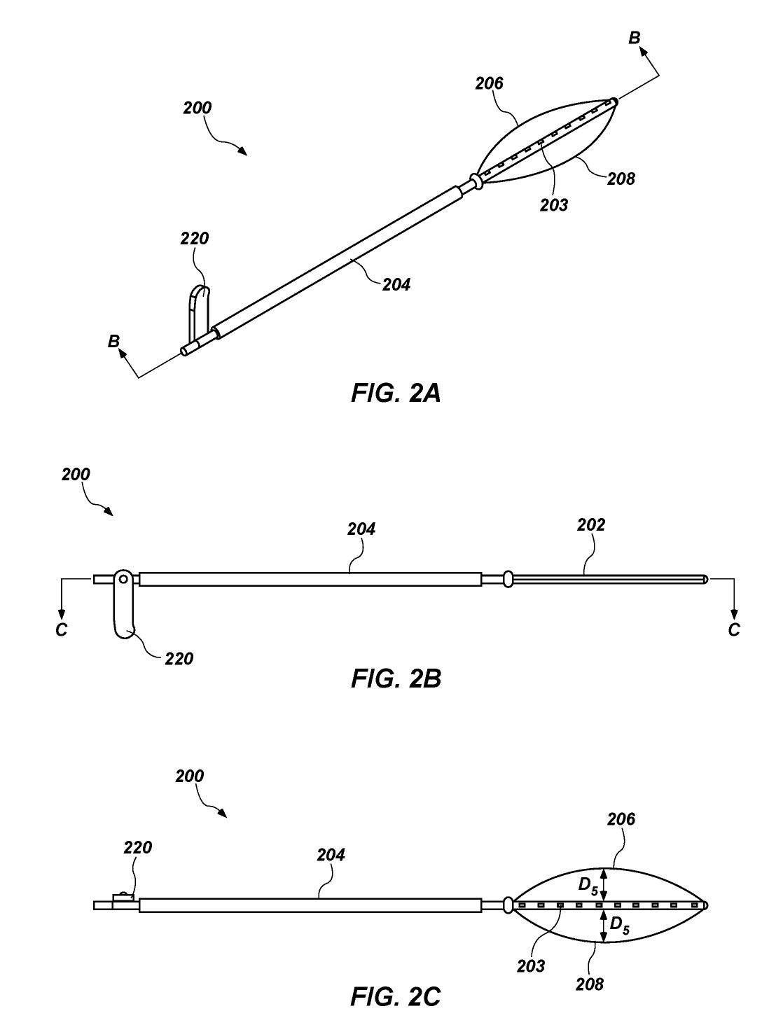

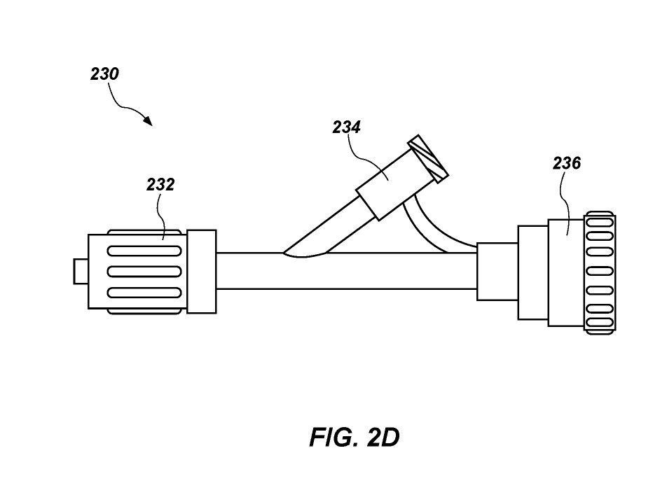

FIG. 2A through FIG. 2C illustrate another embodiment of a device 200 for treating venous incompetence and delivering a treatment fluid to a patient, in accordance with embodiments of the disclosure. FIG. 2A is a simplified perspective view of the device 200 in a contracted configuration (i.e., with a catheter disposed within a sheath of the device). FIG. 2B is a simplified cross-sectional view of the device 200 taken along section line B-B of FIG. 2A. FIG. 2C is a simplified cross-sectional view of the device 200 taken along section line C-C of FIG. 2B. The device 200 may be configured to introduce a treatment fluid to a patient.

With reference to FIG. 2A through FIG. 2C, the device 200 may include a catheter 202 configured to be disposed in a sheath 204 (as illustrated in FIG. 2A). Accordingly, the sheath 204 may be configured to retain the catheter 202. As described above with reference to FIG. 1A through FIG. 1D, the sheath 204 may be configured to move relative to the catheter 202 to remove the catheter 202 from the sheath 204. Responsive to removal of the sheath 204 from over surfaces of the catheter 202, a first expansion element 206 and a second expansion element 208, as described above with reference to the first expansion element 106 and the second expansion element 108, may be exposed. The first expansion element 206 and the second expansion element 208 may comprise the same materials described above with reference to the first expansion element 106 and the second expansion element 108.

The catheter 202 may further include perforations 203 configured to deliver a treatment fluid to a patient when the catheter 202 is disposed in the luminal vessel of the patient. The catheter 202 may also include an opening at the distal end thereof, for introducing a treatment fluid through the opening.

The first expansion element 206 and the second expansion element 208 may be configured to expand and stretch the luminal vessel, as described above with reference to FIG. 1A through FIG. 1D. In some embodiments, a distance D₅ from the catheter 202 to the surface of the first expansion element 206 and the second expansion element 208 when the first expansion element 206 and the second expansion element 208 are expanded may vary along a longitudinal length of the first expansion element 206 and the second expansion element 208. Accordingly, a cross-sectional area of the luminal vessel may vary along a longitudinal length of the catheter 202 proximate the first expansion element 206 and the second expansion element 208 when the first expansion element 206 and the second expansion element 208 are deployed in the luminal vessel.

With continued reference to FIG. 2A through FIG. 2C, the device 200 may further include a lever 220 configured to manipulate a location (e.g., an amount of expansion) of the first expansion element 206 and the second expansion element 208. In some embodiments, the lever 220 may be located at a proximate end of the device 200 and configured to be positioned outside of a patient and manipulated by a medical technician. The lever 220 may be configured to be in operable communication with the first expansion element 206 and the second expansion element 208. In some embodiments, rotation of the lever 220 in a first direction may expand the first expansion element 206 and the second expansion element 208 and rotation of the lever 220 in an opposite, second direction may retract the first expansion element 206 and the second expansion element 208. In some such embodiments, a clinician may operate the lever 220 from, for example, a hub of the catheter 202 located external to the patient. In some embodiments, the lever 220 is configured to receive an end of the first expansion element 206 and the second expansion element 208 and configured to lock the first expansion element 206 and the second expansion element 208 in an expanded position after the first expansion element 206 and the second expansion element 208 are expanded. By way of nonlimiting example, the lever 220 may include or comprise a hemostatic valve. After the first expansion element 206 and the second expansion element 208 are expanded, the hub may be turned to lock the first expansion element 206 and the second expansion element 208 in the hemostatic valve. The hemostatic valve may further include an injection port for introducing the treatment fluid into the device 200.

FIG. 2D is a simplified cross-sectional view of an adapter 230 that may be used in the device 200, in accordance with embodiments of the disclosure. In some embodiments, the lever 220 (FIG. 2A through FIG. 2C) comprises the adapter 230. The adapter 230 may include a first end 232 configured to receive the catheter 202. The first end 232 may comprise a hub configured to receive and operably connect to the catheter 202. Accordingly, the catheter 202 may be in operable communication with the adapter 230 at the first end 232. The adapter 230 may further include a second end 234 located on a side of the adapter 230. The second end 234 may be configured to connect to another device or component. In some embodiments, the second end 234 may include a luer fitting for connecting to, for example, a syringe including the treatment fluid.

The adapter 230 may further include a third end 236 located opposite the first end 232, which may comprise a luer type fitting configured to receive one or more other devices. In some embodiments, the third end 236 may include a valve.

In some embodiments, the adapter 230 comprises a hemostatic valve, such as a rotating hemostatic valve. In some such embodiments, wires comprising the first expansion element 206 and the second expansion element 208 may be located within the adapter 230 and extend through the sheath 204 to the distal end of the device 200. The first expansion element 206 and the second expansion element 208 may be advanced through the catheter 202 through the adapter 230. After the first expansion element 206 and the second expansion element 208 are advanced to a desired location, the hub at the first end 232 may be rotated to lock the first expansion element 206 and the second expansion member 208 within the adapter 230. After the first expansion element 206 and the second expansion element 208 are locked, the treatment fluid may be introduced to the catheter 202, such as through the second end 234.

It is contemplated that in some embodiments, the adapter 230 includes a ratcheting mechanism for advancing and retracting the first expansion element 206 and the second expansion member 208. By way of nonlimiting example, the adapter 230 may include teeth in operable communication with each of the first expansion element 206 and the second expansion element 208 and configured to advance and retract the first expansion element 206 and the second expansion element 208. As one example, the first expansion element 206 and the second expansion element 208 may be advanced or retracted by rotating one or more gears comprising teeth in operable communication with the first expansion element 206 and the second expansion element 208.

In other embodiments, the adapter 230 may include a touey-borst adapter.

In some embodiments, the device 100, 200 may be indexed to facilitate controlled and reproducible exposure along the length of the vein. In some embodiments, the sheath 104, 204 may be indexed relative to the catheter 102, 202 at a proximal location to facilitate adequate exposure of the luminal vessel. For example, the sheath 104, 204 may be indexed incrementally according to the treatment length of the catheter 102, 202. For example, if the length of the catheter 102, 202 including the perforations 103, 203 proximate the first expansion element 106, 206 and the second expansion element 108, 208 is about 5 cm, after the first expansion element 106, 206 and the second expansion element 108, 208 are retracted back into the sheath 104, 204, the sheath 104, 204 may be pulled back (e.g., retracted) relative to the catheter 102, 202 to the next indexed increment and the catheter 102, 202 may be redeployed to treat the next 5 cm segment of the vein. The process may be repeated to treat the entire incompetent vein.

In use and operation, the device 100, 200 may be inserted into a patient at a treatment location, the sheath 104, 204 may be retracted to expose the at least one perforation 103, 203 formed in the wall of the catheter 102, 202 and release the constraints of the first expansion element 106, 206 and the second expansion element 108, 208. A first biasing element (not shown) is a means for causing the first expansion element 106, 208 to expand and a second biasing element (not shown) is a means for causing the second expansion element 108, 208 to expand. The first biasing element and the second biasing element may be a spring or the inherent elastic properties of the first expansion element 106, 206 and the second expansion element 108, 208. Once expanded, the first expansion element 106, 206 and the second expansion element 108, 208 are a means for distorting the luminal vessel.

Once the luminal vessel is distorted, the catheter 102, 202 may be pushed forward to lock the first expansion element 106, 206 and the second expansion element 108, 208 into place. The first expansion element 106, 206 and the second expansion element 108, 208 may distort the luminal vessel from a substantially circular cross-section to a rectangular ribbon. This distortion reduces the cross-sectional area and thus, decreasing the luminal volume while maximizing the surface area of the luminal vessel reducing the volume of chemical required to treat the vessel walls. In addition, stretching the luminal vessel will reduce likelihood of the vessel wall spasming and developing pockets that are not treated by the chemical. Further, the distortion may induce a stretch injury and increase wall tension that may potentiate sclerosant activity on the endothelium.

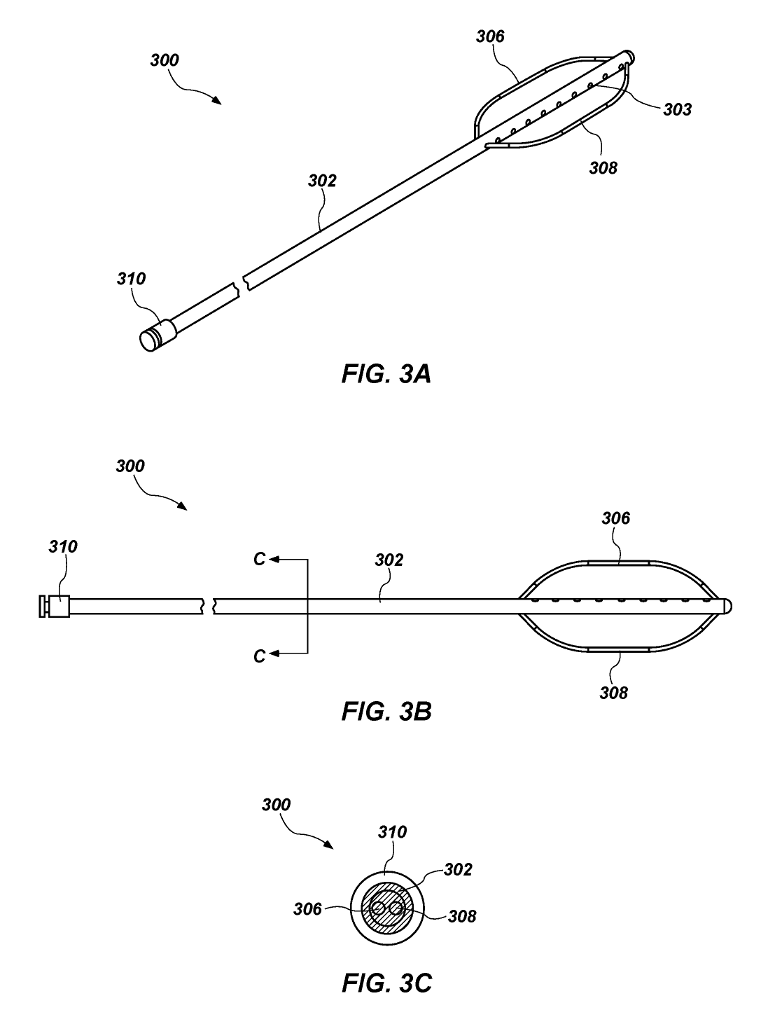

In further embodiments, the device for venous incompetence may not include a sheath housing the expansion elements. FIG. 3A through FIG. 3C are a perspective view, a plan view, and a cross-sectional view of a device 300 for treating varicose veins, in accordance with embodiments of the disclosure. The device 300 may include a catheter 302, which may be substantially the same as the catheters 102, 202 described above with reference to FIG. 1A through FIG. 2C. The catheter 302 may include one or more perforations 303 in sides thereof, at a distal end thereof, or both. In some embodiments, the catheter 302 may comprise a permeable material configured to facilitate flow of a treatment fluid through at least a portion of sidewalls thereof.

With reference to FIG. 3C, the first expansion element 306 and the second expansion element 308 may extend along sidewalls of the catheter 302 along a length thereof. The first expansion element 306 and the second expansion element 308 may each be configured to expand from the catheter 302 at a proximate-most perforation 303 and a distal most perforation 303.

In some embodiments, rotation of a catheter hub 310, may expand the first expansion element 306 and the second expansion element 308. Rotation of the catheter hub 310 in an opposite direction may retract the first expansion element 306 and the second expansion element 308.

In use and operation, the catheter 302 may be advanced to a desired location in a luminal vessel to be treated. After the catheter 302 is placed at a desired location, the catheter hub 310 may be rotated to expand the first expansion element 306 and the second expansion element 308 to stretch the luminal vessel walls and reduce a cross-sectional area of the luminal vessel. After the expansion elements 306, 308 are expanded, the treatment fluid may be introduced into the luminal vessel through the perforations 303. After treatment of the location, the catheter hub 310 may be rotated to contract the first expansion element 306 and the second expansion member 308, the catheter 302 may be moved to a different location to be treated, and the process may be repeated to treat the different location.

In some embodiments, the catheter 302 may be introduced to the desired location over, for example, a guidewire. In some such embodiments, a guidewire may be introduced into the vessel to a desired location to be treated. The catheter 302 may be introduced to the treatment location over the guidewire and the guidewire may be retracted through the catheter 302. Thereafter, the first expansion element 306 and the second expansion element 308 may be expanded to distort the luminal vessel and the incompetent vein may be treated.

In some embodiments, the catheters 102, 202, 302 described above may be configured as multi-lumen catheters, such as dual lumen catheters. In some such embodiments, each lumen of the catheter 102, 202, 302 may be configured to deliver a different treatment fluid (e.g., sclerosing agent, drugs, etc.) to the treatment location.

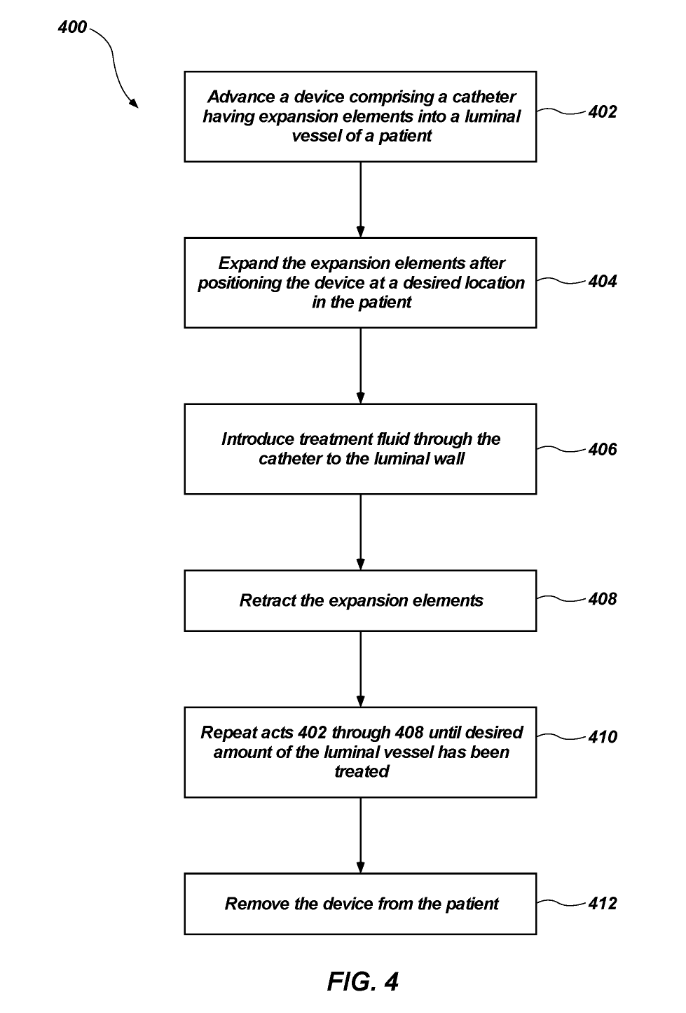

FIG. 4 is a simplified flow diagram of a method 400 of treating a patient using one of the devices 100, 200, 300 described above. The method 400 includes act 402 including advancing a device comprising a catheter having expansion elements into a luminal vessel of a patient; act 404 including expanding the expansion elements after positioning the device at a desired location in a patient; act 406 including introducing treatment fluid through the catheter to the luminal wall; act 408 including retracting the expansion elements; act 410 including repeating acts 402 through 408 until a desired amount of the luminal vessel has been treated; and act 412 including removing the device from the patient.

Act 402 includes advancing a device comprising a catheter having expansion elements into a luminal vessel of a patient. The device may be substantially the same as one of the devices 100, 200, 300 described above with reference to FIG. 1A through FIG. 3C. In some embodiments, the device may be introduced into the patient with, for example, an introducer sheath.

Act 404 includes expanding the expansion elements after positioning the device at a desired location in the patient. In some embodiments, the device is advanced to a target location within the patient. After the device is positioned at a desired location, the expansion elements may be expanded to contact the inner walls of the luminal vessel. In some embodiments, a sheath of the device is moved relative to a catheter disposed within the sheath to expose the catheter. Movement of the sheath relative to the catheter may, in some embodiments, release the expansion elements which may be biased to expand outward from the catheter. In other embodiments, the device may include a lever or other actuating means to expand the expansion elements after removing the sheath from the portion of the catheter including the expansion elements. In further embodiments, such as those described above with reference to FIG. 3A through FIG. 3C, the expansion elements may be located in the catheter. Manipulation of, for example, the catheter hub, may expand and contract the expansion elements.

Expansion of the expansion elements may stretch the walls of the luminal vessel in such a manner that the cross-sectional area of the luminal vessel is reduced. As described above, in some embodiments, the luminal vessel may exhibit a substantially rectangular or an elongated elliptical cross-sectional shape.

Act 406 includes introducing treatment fluid through the catheter to the luminal wall. The treatment fluid may be introduced through one or more perforations in the catheter located at a longitudinal location of the catheter corresponding to a location of the expansion elements. In some embodiments, the treatment fluid may comprise a sclerosing agent. By way of nonlimiting example, the treatment fluid may comprise alcohols such as ethanol, polidocanol (POL), sodium tetradecyl sulphate (STS), hypertonic saline, glycerin, chromated glycerin, another sclerosing agent, or combinations thereof.

Act 408 includes retracting the expansion elements. In some embodiments, retracting the expansion elements may include manipulating a lever to which the expansion elements in operable communication with. In other embodiments, the expansion elements may be retracted by sliding the sheath over the catheter, wherein the sheath houses the expansion elements.

Act 410 includes repeating acts 402 through 408 until a desired amount of the luminal vessel has been treated. For example, the sheathed catheter may be moved by a distance corresponding to the length of the treatment tip (i.e., the length of the expansion elements), the expansion elements may be expanded, the treatment fluid may be introduced to the luminal vessel, and the expansion elements may be retracted. Act 410 may be performed a desired number of times until a sufficient amount of the luminal vessel has been treated.

Act 412 includes removing the device from the patient, which may comprise retracting the catheter and the device from the patient.

Since the expansion elements reduce the volume of the luminal vessel (e.g., the incompetent vein), an operator may not be required to apply external pressure to a patient during treatment, as is often required when using conventional treatment devices. Stated another way, the device may be configured to internally expand and apply internal pressure to reduce a cross-sectional area of the luminal vessel. In addition, since the expansion elements may comprise a thin wire shape, expansion of the expansion elements may not occlude blood flow through the luminal vessel, but may facilitate introduction of the treatment fluid into a reduced volume of the luminal vessel. In addition, expansion of the expansion elements may stretch and smooth the luminal walls, reducing any pockets that may form by spasming of the vessel during administration of the treatment fluid. Stretching and smoothing the luminal walls may increase an effectiveness of the treatment of the luminal vessel.

Although FIG. 1A through FIG. 4 have been described as including a single catheter for distorting the luminal vessel and for introducing the treatment chemical (e.g., the sclerosing agent), the disclosure is not so limited. In other embodiments, a first catheter including, for example, first and second expansion elements may be introduced into the luminal vessel. After placing the first catheter at a desired location to be treated, the first and second expansion elements may be expanded to distort the luminal vessel. When the luminal vessel is distorted by the first catheter, a second catheter including at least one of one or more perforations, an opening at a distal end, or a material permeable to a treatment fluid may be introduced through the first catheter. In some embodiments, a distal portion of the second catheter may extend beyond a distal portion of the first catheter. In other embodiments, a distal portion of the second catheter may extend to a location proximate the first and second expansion elements of the first catheter. The treatment fluid may be introduced through the second catheter while the first catheter is in an expanded configuration. In some embodiments, after the treatment fluid is introduced, the second catheter may be removed from the luminal vessel through the first catheter. In other embodiments, the first catheter may be retracted and moved to a different location to be treated and expanded to distort the luminal vessel at the different location. The second catheter may be moved through the first catheter to different location and the treatment fluid may be introduced to the luminal vessel through the second catheter. The process may be repeated until a desired amount of the luminal vessel has been treated.

The devices described herein may be embodiment in other specific forms without departing from their spirit or characteristics. The described embodiments are to be considered in all respects only as illustrative and not restrictive.

The various components of the devices described above may be constructed generally out of any materials suitable for insertion into a patient.

Many modifications and other embodiments of the embodiments described and illustrated herein will come to mind to one skilled in the art to which the disclosure pertains and having the benefit of the teaching presented in the foregoing descriptions and the associated drawings. Therefore, it should be understood that the disclosure is not to be limited to the specific embodiments disclosed and that modifications and other embodiments are intended to be included within the scope of the appended claims. Although specific terms are employed herein, they are used in a generic and descriptive sense only, and not for purposes of limitation.

While the disclosure is susceptible to various modifications and alternative forms, specific embodiments have been shown by way of example in the drawings and have been described in detail herein. However, the disclosure is not intended to be limited to the particular forms disclosed. Rather, the disclosure is to cover all modifications, equivalents, and alternatives falling within the scope of the disclosure as defined by the following appended claims and their legal equivalents.

What is claimed is:

1. A device for treating venous incompetence, the device comprising:
a catheter disposed within a sheath; and
an expansion element disposed within the sheath in a compressed state and attached to the catheter, the expansion element configured such that when the sheath is retracted within a luminal vessel the expansion element expands to contact and laterally deform a wall of the luminal vessel to reduce a cross-sectional area of the luminal vessel.

2. The device of claim 1, wherein the catheter comprises at least one perforation configured to deliver a treatment fluid to the luminal vessel when the expansion vessel is expanded and in contact with the wall of the luminal vessel.

3. The device of claim 2, wherein the at least one perforation comprises a plurality of perforations.

4. The device of claim 1, wherein the expansion element comprises one or more expansion elements.

5. The device of claim 1, wherein the expansion element comprises two expansion elements.

6. The device of claim 5, wherein the two expansion elements comprise:
a first expansion element attached to the catheter; and
a second expansion element attached to the catheter at a location circumferentially offset from a location at which the first expansion element is attached to the catheter by about 180°.

7. The device of claim 6, wherein the catheter comprises at least one perforation, the at least one perforation circumferentially offset from the first expansion element and the second expansion element.

8. The device of claim 5, wherein the first expansion element and the second expansion element are configured to form an elongated elliptical cross-sectional shape or a rectangular cross-sectional shape of the luminal vessel when expanded.

9. The device of claim 5, wherein the first expansion element and the second expansion element are configured to contact the luminal vessel at only two points of a cross-section of the luminal vessel.

10. The device of claim 5, wherein a distance between the catheter and the first expansion element is substantially constant along a majority of a length of the first expansion element.

11. The device of claim 1, wherein the expansion element comprises a spring.

12. A method of treating venous incompetence, the method comprising:
inserting a device into a luminal vessel, the device comprising:
a catheter disposed within a sheath;
an expansion element disposed within the sheath in a compressed state;
placing the catheter at a treatment location;
retracting the sheath to release the expansion element and contact an interior of the luminal vessel to distort the luminal vessel and reduce a cross-sectional area thereof; and
passing a chemical through perforations in the catheter to treat the luminal vessel.

13. The method of claim 12, wherein passing a chemical through perforations in the catheter comprises passing the chemical through perforations that are circumferentially offset from the expansion element.

14. The method of claim 12, wherein releasing the expansion element comprises releasing two expansion elements, each expansion element of the two expansion elements attached to the catheter about 180° from each other.

15. The method of claim 12, wherein reducing a cross-sectional area of the luminal vessel comprises forming an elongated elliptical cross-sectional area or a substantially rectangular cross-sectional area with the expansion element.

16. A device for treating venous incompetence, the device comprising:
a catheter having perforations in a wall thereof, the perforations located at a distal end of the catheter; and
an expansion element comprising a first expansion element and a second expansion element attached to the catheter and housed within the catheter in an unexpanded configuration, the first expansion element and the second expansion element configured to expand to contact a wall of a luminal vessel in which the device is placed, wherein contact of the wall of the luminal vessel by the first expansion element and the second expansion element reduces a cross-sectional area of the luminal vessel.

17. The device of claim 16, further comprising a sheath disposed around the catheter, wherein the first expansion element and the second expansion element each comprise a wire.

18. The device of claim 17, wherein the expansion element consists of the first expansion element and the second expansion element.

19. The device of claim 16, further comprising a lever in operable communication with the expansion element, the lever located at a proximal end of the catheter and configured to expand and retract the expansion element.

20. The device of claim 16, wherein the expansion element is configured such that a major axis of the luminal vessel has a dimension greater than at least about three times a dimension of a minor axis of the luminal vessel when the expansion element is expanded.

21. A method of treating venous incompetence, the method comprising:
inserting a catheter into a luminal vessel to a treatment location;
distorting the luminal vessel to reduce a cross-sectional area thereof at the treatment location; and
introducing a chemical through the catheter to the reduced cross-sectional area of the luminal vessel.
